(19) United States
(12) Patent Application Publication
(10) Pub. No.: US 2015/0351950 A1
(43) Pub. Date: Dec. 10, 2015

(54) ORAL SECUREMENT APPLIANCE

(71) Applicant: Insono Therapeutics, Inc, East Palo Alto, CA (US)

(72) Inventors: Anant V. HEGDE, Hayward, CA (US); Kasey K. LI, Palo Alto, CA (US)

(73) Assignee: Insono Therapeutics, Inc, East Palo Alto, CA (US)

(21) Appl. No.: 14/300,874

(22) Filed: Jun. 10, 2014

(51) Int. Cl. A61F 5/37

(52) U.S. Cl. CPC A61F 5/37

(57) ABSTRACT

An oral securement appliance is described having a support frame defining a first receiving channel, a distal end of the support frame defining a first opening therethrough, and a valve positioned within or through the first opening. An outer layer may be positioned within the first receiving channel and define a second receiving channel, a distal end of the outer layer defining a second opening in fluid communication with the valve. The device further includes a shell assembly and a cuff member positioned within the second receiving channel and defines a third receiving channel configured for receiving a tongue of the subject. At least an anterior portion of the subject's tongue is inserted within the third receiving channel of the cuff member and a negative pressure drawn within the third receiving channel via the valve such that the cuff member is secured around a periphery of the tongue.

### FIELD OF THE INVENTION

The present invention relates to apparatus and methods for treating breathing disorders by restraining or maintaining a position of a subject's tongue relative to the subject's mouth. More particularly, the present invention relates to apparatus and methods for restraining or maintaining a position of the subject's tongue relative to the mouth for the treatment of various disorders such as snoring, obstructive sleep apnea (OSA), chronic obstructive pulmonary disease (COPD), asthma, etc.

### BACKGROUND OF THE INVENTION

The elimination of snoring and the various degrees of hypopnea and apnea have been attempted because of their various effects on the body. Such conditions typically occur due to pathological disturbances during sleep. One of the reasons of sleep disturbance is due to the tongue relaxing to varying degrees. When fully awake, the tongue has a normal tone and position within the mouth as air passes in and out of the lungs during respiration. Patency of the airway passage between the posterior wall of the pharynx and the base of the tongue is typically maintained for a normal unobstructed airway when the tongue is retained in its natural position.

However, during sleep, as the tongue becomes lax it may collapse against the posterior wall of the pharynx not only causing snoring, but also obstructing the passage of air to the lungs. When the tongue is in its relaxed and collapsed state, the base of the tongue pushes against the soft palate and also obstructs the airway passage.

Conditions such as sleep apnea not only causes drops in the blood oxygenation level, but and may also adversely affect the heart by increasing blood pressure and pulse rate. Many aspects of a person's quality of life, such as a person's physical and emotional health, are affected by obstructive sleep apnea.

A number of conventional treatments exist. For example, conventional treatments such as mask and nasal continuous positive airway pressure (CPAP) devices are typically utilized but compliance is poor and may cause discomfort in the user. Other treatments such as tontme retaining devices are typically made of soft plastic and utilize a tongue-shaped cavity to hold and maintain the tongue in a forward position. Such devices physically clamp onto the tongue using a mechanical grip or retain a portion of the tongue by utilizing a suction device. However, these methods may generally fail due to discomfort by having the tongue protruded at all times even when the patient is awake and/or breathing normally. Moreover, the tongue may not be secured by the suction device at all times.

Accordingly, there is a need for a system which maintains the patency of a person's airways during sleep and which is comfortable enough and easy to use to facilitate compliance by the user.

### BRIEF SUMMARY OF THE INVENTION

An oral device which may be used to treat disordered breathing, such as snoring or obstructive sleep apnea (OSA), may utilize a housing which is designed to rest on the patient's upper and lower lip junctions while remaining secured at least to an anterior portion of a patient's tongue. Such a device may incremental advance the patient's tongue relative to the mouth by varying the level of suction imparted upon the tongue. Yet this device may allow for the patient to swallow without dislodging the tongue from the device.

Generally, the oral device may comprise a support frame defining a first receiving channel and having at least one projection which extends at an angle relative to the device for placement against a lip junction of a subject, a distal end of the support frame defining a first opening therethrough, a valve positioned within or through the first opening defined by the support frame, an outer layer positioned within the first receiving channel and defining a second receiving channel, a distal end of the outer layer defining a second opening in fluid communication with the valve, a shell assembly positioned along the second receiving channel or integrated with the outer layer and further defining a third opening in fluid communication with a channel defined at least partially through the shell assembly, the channel further defining one or more perforations along a proximal concave surface of the shell assembly, wherein the one or more perforations are in fluid communication with the valve via the third opening, and a cuff member positioned within the second receiving channel and defining a third receiving channel configured for receiving a tongue of the subject, the cuff member further defining one or more cuff perforations such that the third receiving channel is in fluid communication with the valve via the one or more cuff perforations.

In use, the oral device may be utilized by inserting at least an anterior portion of a tongue within the third receiving channel of the cuff member, and drawing a negative pressure within the third receiving channel via the valve such that the cuff member is secured around a periphery of the tongue.

The oral securement device may be secured to the patient's tongue via a suction force which may be drawn through the device and within a receiving channel for positioning of the tongue within. For instance, the tongue can be maintained in place relative to the patient's mouth or drawn anteriorly in increments depending upon the amount of suction pressure imparted upon the tongue.

The device may be gently adhered to the patient's tongue by a suction force initially drawn through the device via a suction device (e.g., syringe, pump, etc.) prior to the patient falling asleep and the suction device may be removed from the oral device leaving just the oral device for maintaining the position of the patient's tongue. Because the oral device maintains the suction force against the tongue, a continuous pump or suction device is not required. Additionally, because the device may rest upon the lips and lip junction of the patient, there is no need for any retaining platform for placement against or upon the teeth or dentition within the patient's mouth.

The oral device may be configured into different sizes (e.g., small, medium, medium plus, large, extra-large, etc.) to accommodate the different size anatomies of patients. Generally, the oral device may comprise a support frame which houses or supports each of the components of the device. The support frame may incorporate a valve at a distal end of the frame which may taper outwardly to form two opposed resting projections which extend proximally at an angle relative to a centerline of the device. The resting projections may flare at opposite angles for resting upon the lip junctions of the patient. The support frame may form a receiving channel within and which may hold an outer layer (e.g., fabricated of silicone) which in turn further defines a receiving channel for securing a cuff member or inner layer (e.g., also fabricated of silicone). The cuff member may extend proximally from the support frame to form a peripheral lip which also defines an opening into which the tongue may be inserted at least partially for securement to the device.

When a suction pump, such as a syringe, is connected to the valve air may freely flow through a diaphragm within the valve but when the pump is decoupled from the valve, the internal diaphragm may seal the port to maintain the pressure differential. For instance, a volume of air (e.g., up to 8 cc of air) may be drawn through a conventional medical syringe fluidly coupled to the valve for drawing a sufficient suction pressure.

The support frame may be fabricated from various materials to form a structurally rigid shell (e.g., ABS or rigid plastics such as polycarbonate, acrylic, polystyrene, etc.) and over molded using a relatively softer material (e.g., Santoprene™ thermoplastic elastomer (TPE) or soft materials such as silicone, polyurethane, C-flex®, Versaflex™, other elastomeric materials, etc.). With the support frame tapering outward, a support member may extend between apposed portions of the support frame to provide for structural support on either side of the frame to prevent the frame from opening apart particularly when an axial load is applied by the tongue when secured to the device. Moreover, the resting projections may be formed of the same material as the rest of the frame and they may be coated with relative soft materials (e.g., foam, etc.) for added comfort to the user.

The valve may be attached to a valve stop positioned at the distal end of the support frame and which may define a valve opening through which air may flow. The support frame may further define a support frame receiving channel within which the outer layer may be inserted. The outer layer itself may be formed separately from or integrally with a shell assembly formed from shell members to create a relatively soft layer (e.g., Santoprene™ thermoplastic elastomer (TPE) or soft materials such as silicone, polyurethane, C-flex®, Versaflex™, other elastomeric materials, etc.) which is securely positioned within the support frame. If the outer layer is formed separately from the shell assembly, the outer layer may be inserted within the support frame receiving channel and the shell assembly may be inserted within a receiving channel defined by the outer layer and the cuff member may then be inserted within the receiving channel of the outer layer. Alternatively, if the outer layer is formed integrally with the shell assembly, the outer layer itself may be formed, for instance, by dipping the shell assembly into a polymeric substrate such as silicone or by insert molding. In either case, the formed outer layer may have a material durometer hardness of, e.g., 10 Shore A to 50 Shore A.

The may be formed by two halves (e.g., molded from a rigid plastic such as acrylic material, ABS, polycarbonate, polystyrene, etc.) to create a shell which provides structural integrity to the oral device and further houses the cuff which is secured to the tongue. The shell assembly may form a tapered member which extends from a channel opening while defining a cuff member receiving channel which is defined concavely along the length of the shell assembly to accommodate for positioning of the tongue. The shell assembly may further define a channel which extends at least along a proximal section of the assembly and one or more openings or perforations which extend along a proximal surface of the channel to within the cuff member receiving channel Hence, the channel opening may be in fluid communication within the assembly through the channel and through openings or perforations to the cuff member receiving channel.

The outer layer and the shell assembly may be fluidly coupled at their distal ends to the valve through the support frame via a luer fitting (e.g., BDMFTLL-81, Value Plastics, Inc.) which may be attached to the channel openings defined in the shell assembly and to distal opening defined through the outer layer either by bonding the luer fitting (e.g., cyanoacrylate adhesive or solvents like Acetone, Methyl ethyl ketone (MEK), etc.) or by molding the luer fitting onto the shell assembly. In either case, the luer fitting may form an airtight lumen with the valve such that when the cuff member is inserted within the receiving channel defined by outer layer, the exterior of the cuff member is in fluid communication through the luer fitting and through the valve.

The cuff member may be formed by injection molding a relatively soft material such as silicone having a durometer hardness of, e.g., 10 Shore A to 50 Shore A. The cuff member may define a tapered distal portion which is extends proximally in a relatively flattened shape to form a proximal portion which further extends proximally to form a projecting lip around a periphery of the cuff member. The cuff member may further define the proximal opening into which the tongue may be inserted at least partially for securement to the device. One or both of the flattened surfaces of the distal portion may define one or more perforations which extend through the wall of the cuff member to allow for passage of air from within the opening to an exterior of the member to allow for the application of a vacuum force within the opening. The size (diameter) of each perforation may be 0.050 in. or less and the perforations may be uniformly located over the flattened surfaces or selectively positioned depending upon the desired suction pattern.

The cuff member may be positioned within the receiving channel defined by the outer layer and secured or bonded to the shell assembly, e.g., via an adhesive such as silicone RTV. Alternatively, the cuff member may be secured to the shell assembly by simply insert molding using silicone.

In one example for use of the oral device, at least the anterior portion of the patient's tongue may be inserted within the opening of the cuff member, e.g., prior to the patient falling asleep. A suction unit (such as syringe, pump, etc.) may be fluidly coupled to the valve either directly or via a fluid line. The suction unit may be actuated to withdraw a volume of air (e.g., up to 8 cc of air if using a syringe) to create a desired vacuum level by drawing the air from within the opening of the cuff member, through the one or more perforations defined through the cuff member, through distal opening of outer layer, through the openings or perforations and into the channel and through the channel opening of the shell assembly, through the luer fitting, and finally through the valve. The proximal portion of the cuff member may adhere around the surface of the tongue so long as the negative pressure is maintained within the cuff member. With the negative pressure created, the suction unit may be removed from the valve without disrupting the vacuum force.

The patient may leave the oral device in place over the mouth as the projections come to rest against the patient's lip junctions. Hence, the tongue may be maintained in position relative to the patient's mouth by the oral device to prevent the tongue (particularly the base of the tongue) from collapsing posteriorly. When the patient awakes or if the oral device requires removal for any reason, the syringe or suction unit may be reattached and a positive pressure may be introduced through the valve to release the tongue from the cuff member. Alternatively, a pressure release may be integrated into the valve or elsewhere on the oral device to allow for the flow of air into the oral device.

### BRIEF DESCRIPTION OF THE DRAWINGS

FIG. 1 shows a perspective assembly view of an oral securement device for maintaining or restraining a subject's tongue relative to their mouth.

FIG. 2 shows an exploded assembly view of the oral securement device.

FIG. 3 shows a perspective view of a support frame for the oral securement device.

FIGS. 4A and 4B show perspective views of a shell assembly within the oral securement device.

FIG. 5 shows a perspective view of a cuff member which functions as an inner layer within the oral securement device.

FIG. 6 shows a perspective view of an oral securement device in use while positioned within the mouth of a patient.

### DETAILED DESCRIPTION OF THE INVENTION

In certain breathing disorders, particularly sleep disordered breathing disorders such as snoring or obstructive sleep apnea (OSA), the subject's tongue typically collapses against the posterior wall of the pharynx obstructing the breathing airway. Treatment for such breathing disorders may generally involve maintaining a position of the tongue or protruding the tongue anteriorly relative to the subject's jaw to prevent or inhibit collapse of tissue against the phymgeal wall.

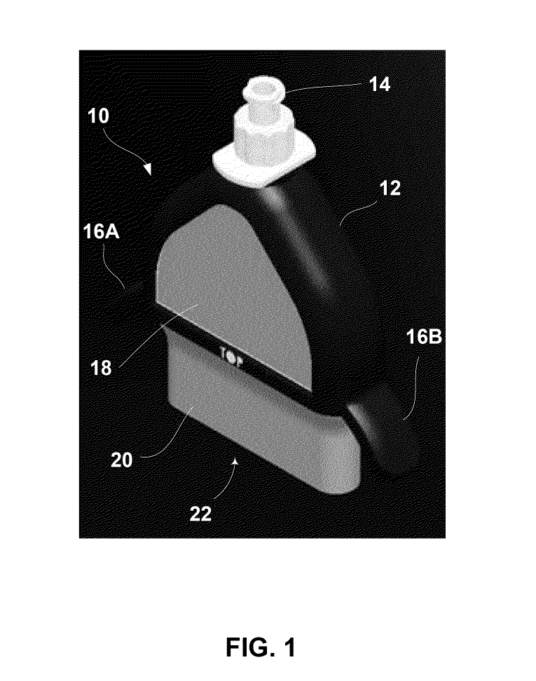

One such device is shown in the perspective view of FIG. 1 which illustrates an assembled oral securement device 10 which is designed to rest on the patient's upper and lower lip junctions while remaining secured at least to an anterior portion of a patient's tongue.

The oral securement device 10 may be secured to the patient's tongue via a suction force which may be drawn through the device and within a receiving channel for positioning of the tongue within. For instance, the tongue can be maintained in place relative to the patient's mouth or drawn anteriorly in increments anywhere from, e.g., 2 to 5 mm, depending upon the amount of suction pressure imparted upon the tongue.

With the tongue securely held within the device 10, a position of the tongue relative to the patient's mouth may be protruded or maintained to prevent a posterior collapse of the tongue against the pharyngeal wall particularly when the patient falls asleep. The device 10 may be gently adhered to the patient's tongue by a suction force initially drawn through the device 10 via a suction device (e.g., syringe, pump, etc.) prior to the patient falling asleep and the suction device may be removed from the oral device 10 leaving just the oral device 10 for maintaining the position of the patient's tongue. Because the oral device 10 maintains the suction force against the tongue, a continuous pump or suction device is not required. Additionally, because the device 10 may rest upon the lips and lip junction of the patient, there is no need for any retaining platform for placement against or upon the teeth or dentition within the patient's mouth.

Moreover, the oral device 10 may incorporate components or be used with the various features and methods of other oral applications as shown and described in each of the following patents and patent applications: U.S. Pat. No. 7,607,439; U.S. Pat. No. 8,028,705; U.S. Pat. No. 8,347,890; U.S. Pat. No. 8,613,283; U.S. Pub. 2012/0204886; U.S. Pub. 2010/0288288; U.S. Pub. 2011/0180076; and U.S. Pub. 2014/0130808. Each of these patents and patent applications are incorporated herein by reference in its entirety and for any purpose herewith.

Generally, the oral device 10 may be configured into different sizes (e.g., small, medium, medium plus, large, extra-large, etc.) to accommodate the different size anatomies of patients. The size of the oral device may range from 5 cm to 6 cm in width and from 6 cm to 10 cm in length. Generally, the oral device 10 may comprise a support frame 12 which houses or supports each of the components of the device 10. The support frame 12 may incorporate a valve 14 at a distal end of the frame 12 which may taper outwardly to form at least one or two opposed resting projections 16A, 16B which extend proximally at an angle relative to a centerline of the device 10. The resting projections 16A, 16B may flare at opposite angles for resting upon the lip junctions of the patient. The support frame 12 may form a receiving channel within and which may hold an outer layer 18 (e.g., fabricated of silicone) which in turn further defines a receiving channel for securing a cuff member or inner layer 20 (e.g., also fabricated of silicone). The cuff member 20 may extend proximally from the support frame 12 to form a peripheral lip which also defines an opening 22 into which the tongue may be inserted at least partially for securement to the device 10.

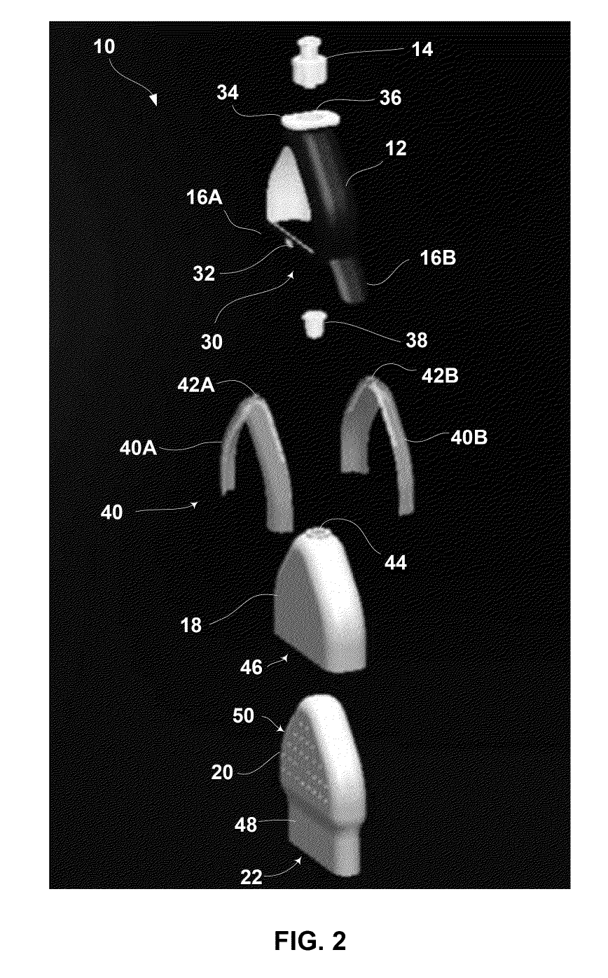

FIG. 2 shows a perspective view of an exploded assembly of the oral device 10. The valve 14 may comprise a uni-directional valve or reflux check valve (e.g., VPS5401036SN, Value Plastics, Inc.) which allows for the maintenance of a negative pressure within the device 10. When a suction pump, such as a syringe, is connected to the valve 14 air may freely flow through a diaphragm within the valve 14 but when the pump is decoupled from the valve 14, the internal diaphragm may seal the port to maintain the pressure differential. For instance, a volume of air (e.g., up to 8 cc of air) may be drawn through a conventional medical syringe fluidly coupled to the valve 14 for drawing a sufficient suction pressure.

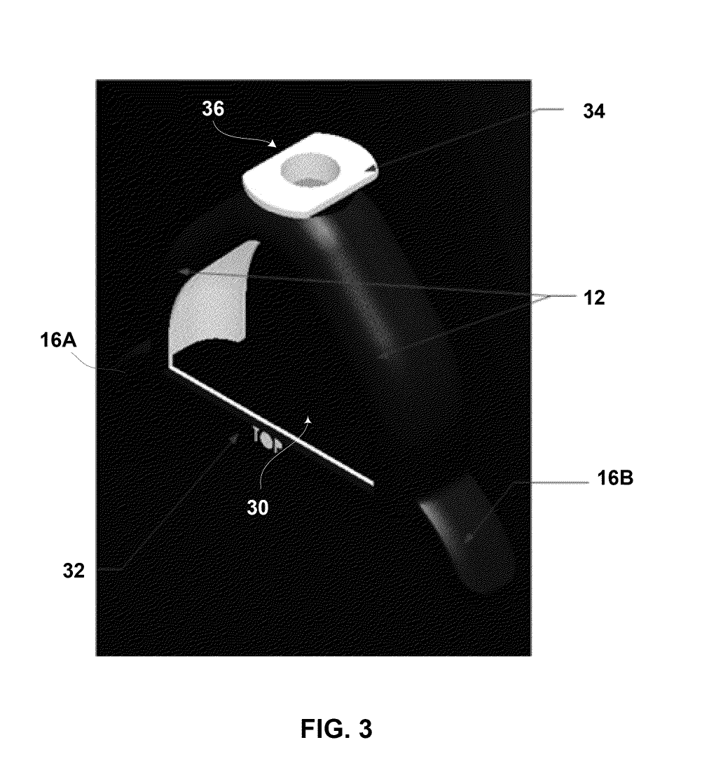

The support frame 12 may be fabricated from various materials to form a structurally rigid shell (e.g., ABS or rigid plastics such as polycarbonate, acrylic, polystyrene, etc.) and over molded using a relatively softer material (e.g., Santoprene™ thermoplastic elastomer (TPE) or soft materials such as silicone, polyurethane, C-flex®, Versaflex™, other elastomeric materials, etc.). A detailed perspective view of the support frame 12 is further shown in FIG. 3. As previously described, the different sizes of the support frame 12 may be color coded to readily differentiate between the various sizes. With the support frame 12 tapering outward, a support member 32 may extend between apposed portions of the support frame 12 to provide for structural support on either side of the frame 12 to prevent the frame 12 from opening apart particularly when an axial load is applied by the tongue when secured to the device 10. Moreover, the resting projections 16A, 16B may be formed of the same material as the rest of the frame 12 and they may be coated with relative soft materials (e.g., foam, etc.) for added comfort to the user.

The valve 14 may be attached to a valve stop 34 positioned at the distal end of the support frame 12 and which may defines a valve opening 36 through which air may flow. The support frame 12 may further define a support frame receiving channel 30 within which the outer layer 18 may be inserted. The outer layer 18 itself may be formed separately from or integrally with a shell assembly 40 formed from shell members 40A, 40B to create a relatively soft layer (e.g., Santoprene™ thermoplastic elastomer (TPE) or soft materials such as silicone, polyurethane, C-flex®, Versaflex™, other elastomeric materials, etc.) which is securely positioned within the support frame 12. If the outer layer 18 is formed separately from shell assembly 40, the outer layer 18 may be inserted within the support frame receiving channel 30 and the shell assembly 40 may be inserted within a receiving channel 46 defined by the outer layer 18 and the cuff member 20 may then be inserted within the receiving channel 46 of the outer layer 18. Alternatively, if the outer layer 18 is formed integrally with the shell assembly 40, the outer layer 18 itself may be formed, for instance, by dipping the shell assembly 40 into a polymeric substrate such as silicone or by insert molding. In either case, the formed outer layer 18 may have a material durometer hardness of, e.g., 10 Shore A to 50 Shore A.

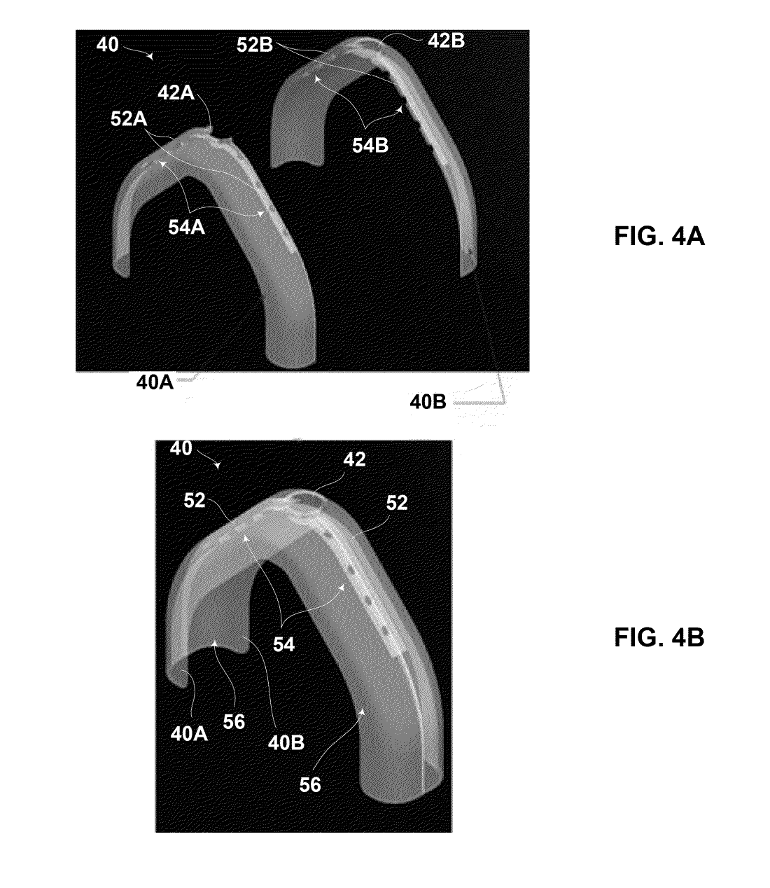

The shell assembly 40 shown in FIG. 2 is further shown in detailed perspective assembly views in FIGS. 4A and 4B. As described, the shell assembly 40 may be formed by two halves 40A, 40B (e.g., molded from a rigid plastic such as acrylic material, ABS, polycarbonate, polystyrene, etc.) to create a shell which provides structural integrity to the oral device 10 and further houses the cuff 20 which is secured to the tongue. The shell assembly 40 may form a tapered member which extends from a channel opening 42 while defining a cuff member receiving channel 56 which is defined concavely along the length of the shell assembly 40 to accommodate for positioning of the tongue. The shell assembly 40 may further define a channel 52 which extends at least along a proximal section of the assembly 40 and one or more openings or perforations 54 which extend along a proximal surface of the channel 52 to within the cuff member receiving channel 56. Hence, the channel opening 42 may be in fluid communication within the assembly 40 through the channel 52 and through openings or perforations 54 to the cuff member receiving channel 56.

In forming the shell assembly 40, the shell halves 40A, 40B may be bonded together using various adhesives (e.g., cyanoacrylate adhesive, solvents like Acetone, MEK,

THF, etc.). Accordingly, the channel opening 42 and channel 52 may be formed by the corresponding openings 42A, 42B and channels 52A, 52B, respectively, defined in each of the shell halves 40A, 40B. Likewise, the openings or perforations 54 may be formed by the openings 54A, 54B defined in each of the shell halves 40A, 40B.

The outer layer 18 and the shell assembly 40 may be fluidly coupled at their distal ends to the valve 14 through the support frame 12 via a luer fitting 38 (e.g., BDMFTLL-81, Value Plastics, Inc.) which may be attached to the channel openings 42A, 42B defined in the shell assembly 40 and to distal opening 44 defined through the outer layer 18 either by bonding the luer fitting 38 (e.g., cyanoacrylate adhesive or solvents like Acetone, Methyl ethyl ketone (MEK), etc.) or by molding the luer fitting 38 onto the shell assembly 40. In either case, the luer fitting 38 may form an airtight lumen with the valve 14 such that when the cuff member 22 is inserted within the receiving channel 46 defined by outer layer 18, the exterior of the cuff member 20 is in fluid communication through the luer fitting 38 and through the valve 14.

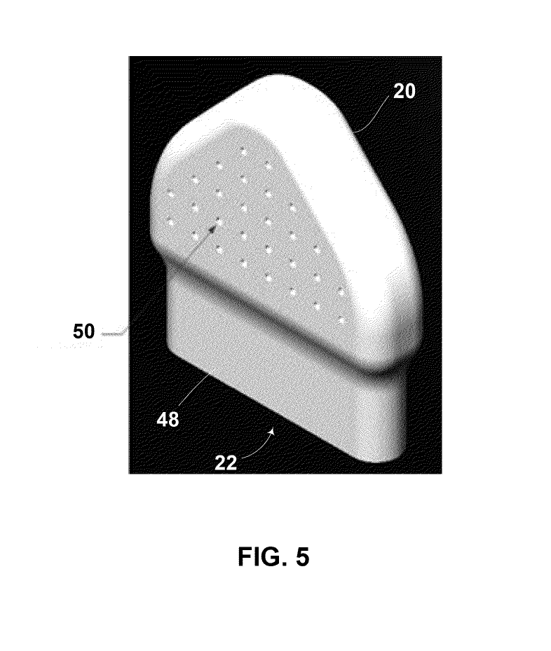

The cuff member 20, shown in FIG. 2 as well as in a detailed perspective view in FIG. 5, may be formed by injection molding a relatively soft material such as silicone having a durometer hardness of, e.g., 10 Shore A to 50 Shore A. The cuff member 20 may define a tapered distal portion which is extends proximally in a relatively flattened shape to form a proximal portion 48 which further extends proximally to form a projecting lip around a periphery of the cuff member 20. The cuff member 20 may further define the proximal opening 22 into which the tontme may be inserted at least partially for securement to the device 10. One or both of the flattened surfaces of the distal portion may define one or more perforations 50 which extend through the wall of the cuff member 20 to allow for passage of air from within the opening 22 to an exterior of the member 20 to allow for the application of a vacuum force within the opening 22. The size (diameter) of each perforation 50 may be 0.050 in. or less and the perforations 50 may be uniformly-located over the flattened surfaces or selectively positioned depending upon the desired suction pattern.

The cuff member 20 may be positioned within the receiving channel 46 defined by the outer layer 18 and secured or bonded to the shell assembly 40, e.g., via an adhesive such as silicone RTV. Alternatively, the cuff member 20 may be secured to the shell assembly 40 by simply insert molding using silicone.

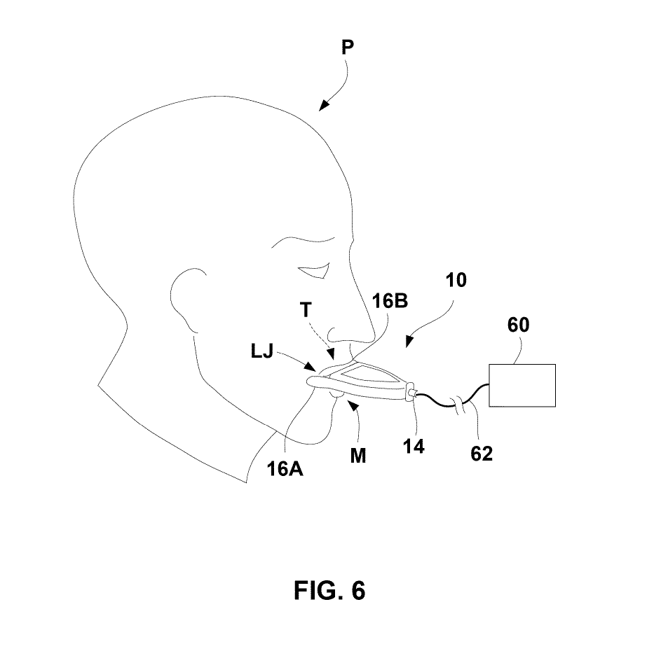

In one example for use of the oral device 10, at least the anterior portion of the patient's tongue may be inserted within the opening 22 of cuff member 20, e.g., prior to the patient P falling asleep. A suction unit 60 (such as syringe, pump, etc.) may be fluidly coupled to the valve 14 either directly or via a fluid line 62, if desired, as shown in the illustration of FIG. 6. The suction unit 60 may be actuated to withdraw a volume of air (e.g., up to 8 cc of air if using a syringe) to create a desired vacuum level by drawing the air from within the opening 22 of cuff member 20, through the one or more perforations 50 defined through the cuff member 20, through distal opening 44 of outer layer 18, through the openings or perforations 54 and into channel 52 and through channel opening 42 of shell assembly 40, through leer fitting 38, and finally through the valve 14. The negative pressure drawn may be maintained at a static pressure level. In other variations, the negative pressure may be continuously or intermittently drawn (e.g., at varying levels) to account for air which may seep into the cuff member due to any uneven adherence to the tongue, although this is unnecessary. The proximal portion 48 of cuff member 20 may adhere around the surface of the tongue T so long as the negative pressure is maintained within the cuff member 20. With the negative pressure created, the suction unit 60 may be removed from the valve 14 without disrupting the vacuum force.

As shown, the patient P may leave the oral device 10 in place over the mouth M as the projections 16A, 16B come to rest against the patient's lip junctions LJ. Hence, the tongue T may be maintained in position relative to the patient's mouth M by the oral device 10 to prevent the tongue (particularly the base of the tongue) from collapsing posteriorly. When the patient P awakes or if the oral device 10 requires removal for any reason, the syringe or suction unit 60 may be reattached and a positive pressure may be introduced through the valve 14 to release the tongue T from the cuff member 20. Alternatively, a pressure release may be integrated into the valve 14 or elsewhere on the oral device 10 to allow for the flow of air into the oral device 10. The oral device 10 may be simply cleaned and reused afterwards.

Moreover, any of the devices, assemblies, methods, etc. may be utilized for a variety of sleep disordered breathing treatment either alone or in combination with other treatment modalities. For example, the devices and methods described may be used along for indications such as snoring, sleep apnea, etc., as well as in combination with other mandibular advancement devices or treatments such as continuous positive pressure (CPAP) therapy to facilitate opening of the airway and reduce the pressure requirements.

While illustrative examples are described above, it will be apparent to one skilled in the art that various changes and modifications may be made therein. Additionally, the devices and methods herein may be utilized to treat other breathing disorders (e.g., chronic obstructive pulmonary disease (COPD), asthma, etc.) Moreover, various apparatus or methods described above are also intended to be utilized in combination with one another, as practicable. The appended claims are intended to cover all such changes and modifications that fall within the true spirit and scope of the invention.

What is claimed is:

1. An oral device, comprising:
a support frame defining a first receiving channel and having at least one projection which extends at an angle relative to the device for placement against a lip junction of a subject, a distal end of the support frame defining a first opening therethrough;
a valve positioned within or through the first opening defined by the support frame;
an outer layer positioned within the first receiving channel and defining a second receiving channel, a distal end of the outer layer defining a second opening in fluid communication with the valve;
a shell assembly positioned along the second receiving channel or integrated with the outer layer and further defining a third opening in fluid communication with a channel defined at least partially through the shell assembly, the channel further defining one or more perforations along a proximal concave surface of the shell assembly, wherein the one or more perforations are in fluid communication with the valve via the third opening; and
a cuff member positioned within the second receiving channel and defining a third receiving channel configured for receiving a tongue of the subject, the cuff member further defining one or more cuff perforations such that the third receiving channel is in fluid communication with the valve via the one or more cuff perforations.

2. The device of claim 1 wherein the support frame further comprises a valve stop positioned at the first opening.

3. The device of claim 1 wherein the support frame further comprises at least two projections which extend at an angle away from one another.

4. The device of claim 1 wherein the support frame is configured to taper from the first opening.

5. The device of claim 1 wherein the valve is configured as a uni-directional or check valve.

6. The device of claim 1 wherein the outer layer is comprised of an elastomeric material.

7. The device of claim 1 wherein the shell assembly comprises a tapered member which extends from the third opening and further defines the concave surface along a length of the shell assembly.

8. The device of claim 1 wherein the cuff member further comprises a proximal portion which extends proximally to for a projecting lip around a periphery of the cuff member.

9. The device of claim 1 wherein the one or more cuff perforations are defined through a wall of the cuff member along one or more flattened surfaces of the cuff member.

10. The device of claim 1 wherein the one or more cuff perforations have a diameter no greater than 0.050 in.

11. A method for maintaining a position of a tongue in a subject, comprising:
providing an oral device having a support frame defining a first receiving channel, a distal end of the support frame defining a first opening tlierethrough, a valve positioned within or through the first opening, an outer layer positioned within the first receiving channel and defining a second receiving channel, a distal end of the outer layer defining a second opening in fluid communication with the valve, a shell assembly positioned along the second receiving channel or integrated with the outer layer and further defining a third opening in fluid communication with a channel defined at least partially through the shell assembly, and a cuff member positioned within the second receiving channel and defining a third receiving channel configured for receiving a tongue of the subject;
inserting at least an anterior portion of a tonaue within the third receiving channel of the cuff member; and,
drawing a negative pressure within the third receiving channel via the valve such that the cuff member is secured around a periphery of the tongue.

12. The method of claim 11 wherein the channel defined through the shell assembly further defines one or more perforations along a proximal concave surface of the shell assembly, and wherein the one or more perforations are in fluid communication with the valve via the third opening.

13. The method of claim 11 Where the cuff member further defines one or more cuff perforations such that the third receiving channel is in fluid communication with the valve via the one or more cuff perforations.

14. The method of claim 11 further comprising positioning one or projections which extend at an angle from the support frame relative to the device against a lip junction of the subject.

15. The method of claim 11 wherein drawing a negative pressure comprises withdrawing a volume of air from within the third receiving channel via a suction apparatus fluidly coupled with the valve.

16. The method of claim 15 further comprising removing the suction apparatus from the valve wherein the negative pressure is maintained.

17. The method of claim 11 wherein drawing a negative pressure comprises withdrawing a volume of air such that a static negative pressure level is maintained within the third receiving channel.

18. The method of claim 11 further comprising introducing a volume of air into the third receiving channel such that the tongue is removed.
